(19) United States
(12) Patent Application Publication
(10) Pub. No.: US 2022/0193091 A1
(43) Pub. Date: Jun. 23, 2022

(54) COMBINED ADMINISTRATION OF DIAZEPAM AND DICLOFENAC FOR THE TREATMENT OF PAIN

(71) Applicant: Mohammad Amin JAHAN PANAH, Dortmund (DE)

(72) Inventor: Abbas JAHAN PANAH, Dortmund (DE)

(73) Assignee: Mohammad Amin JAHAN PANAH, Dortmund (DE)

(21) Appl. No.: 17/610,030

(22) PCT Filed: May 7, 2020
(86) PCT No.: PCT/DE2020/100384

§ 371 Date: Nov. 9, 2021

(30) Foreign Application Priority Data
May 9, 2019 (DE) 10 2019 112 208.2
Mar. 6, 2020 (DE) 10 2020 106 223.0

(51) Int. Cl. A61K 31/5513; A61K 31/196; A61P 29/00

(52) U.S. Cl. CPC A61K 31/5513; A61P 29/00; A61K 31/196

(57) ABSTRACT

A pharmaceutical composition consisting of a combined preparation of diazepam and diclofenac sodium for use as a combined preparation, in the form of tablets, capsules, injection, infusion, drinking ampoules or drops, for the combined administration of diazepam and diclofenac sodium for the short-term specific treatment of pain based on the indication of disc herniation, lower back pain, lumboischialgia, dorsalgia, cervicalgia, cervical neuralgia, sciatica, chronic joint pain, migraine, headache, joint pain, myocardial infarction, post-operative pain, broken bones, burns, tumour pain or tumour-related pain, rheumatism, toothache, pain caused by drug withdrawal, pain that cannot be localised or pain without a known cause, pain caused by fasciae and conjunctive tissue, phantom pain following amputations, combinations of the aforementioned pains and other pains.

The invention relates to a pain medicament consisting of the two components diazepam and diclofenac sodium, for use for migraines, acute severe back pain, significant joint complaints, and for a multitude of other pain states. It is very powerful and is a substitute for morphine treatments. Short-term or long-term administration is possible and it should not be taken with other medicines. The two components have long been known and are widely used.

Diazepam was invented in 1950 and has been marketed under the trade name Valium since 1963. In addition to its use for the symptomatic treatment of acute states of stress, agitation, and anxiety, diazepam is used in premedication before surgical and diagnostic interventions. It is also used as a muscle relaxant and as an emergency therapeutic for the prophylaxis and anticonvulsant treatment of epileptic grand mal seizures and for the treatment of febrile seizures in children. These uses are well known and it is possible to read up on them in Wikipedia at any time. Uses for migraines, acute severe back pain and significant joint complaints are not known for diazepam alone.

A multitude of interactions are generally known. Drugs that affect the brain, such as tranquilizers, soporifics, drugs to treat depression, various analgesics, anticonvulsants such as antiepileptics, and muscle relaxants, and also certain drugs for gastric ulcers, tuberculosis, fungal infections, asthma or for alcohol cessation and diazepam all mutually influence one another's effects. When combining diazepam with other centrally acting substances such as alcohol, neuroleptics, anxiolytics, sedatives, antidepressants, hypnotics, anticonvulsants, narcotic analgesics, anesthetics and sedating antihistamines, account must be taken of the possibility of mutual potentiation of their effects. However, no potentiation has thus far come to light with diclofenac sodium, moreover diclofenac sodium does not belong to any of the abovementioned substance classes.

Diazepam is also known to have many side effects. Those reported include fatigue, severe daytime sedation, drowsiness, somnolence, lassitude, dizziness, headache, ataxia, prolonged reaction times, confusion, anterograde amnesia, hangover effects such as impaired concentration and residual fatigue, impaired reactions. Antidotes such as caffeine are often taken to counter these side effects.

Diazepam is broken down by the cytochrome P450 enzyme system. Inhibitors of this enzyme system result in slower breakdown of diazepam in tandem with prolongation or potentiation of its effect. In addition, diazepam potentiates the effect of other muscle relaxants and also the effect of nitrous oxide and analgesics. The combined use of diazepam and omeprazole, cimetidine, ketoconazole, fluvoxamine, and fluoxetine should be avoided, since these substances slow down the breakdown of diazepam. This is important, since omeprazole is often used to reduce the disruptive effects on the stomach of painkillers, including inter alia diclofenac sodium.

Diazepam is also generally known to act as an allosteric modulator of the GABA_{A} receptor and to potentiate the inhibitory effect of the neurotransmitter GABA. Diazepam binds here as an agonist to the benzodiazepine binding site of this receptor, thereby causing it to undergo conformational change. This increases receptor sensitivity to GABA. Intensified GABA activity results in an increased opening rate in the chloride channel and thus in an increased influx of chloride ions into the cell. The increase in the intracellular chloride concentration results, as a consequence of hyperpolarization, in a reduction in excitability of the cell.

In contrast, the effects of diclofenac, which was invented in 1965, are based on inhibition of cyclooxygenase and consequent reduction in prostaglandin synthesis. This is brought about through inhibition of cyclooxygenases COX-1 and COX-2, which are as a result no longer able to produce proinflammatory prostaglandins. It is possible that diclofenac is directly involved in lipoxygenase metabolism and suppresses the formation of leukotrienes. The analgesic action of diclofenac is heightened by piperine, a constituent of pepper. Diclofenac is available in the form of the sodium or potassium salt. The present invention relates only to the sodium salt diclofenac sodium.

The two mechanisms of action of diazepam and diclofenac are sufficiently different that a synergetic effect cannot seem obvious.

Many undesirable effects have come to light for diclofenac too. The most common are digestive complaints, fluid retention, hypertension, rash, headache, dizziness, and drowsiness.

Diclofenac is one of the nonsteroidal anti-inflammatory drugs, or NSAIDs for short. This technical term means that it is an anti-inflammatory agent, but not a cortisone-type product or a morphine derivative. Diclofenac has an analgesic and anti-inflammatory action and also brings down fever.

The use of diclofenac to treat migraines is also known. For instance, diclofenac potassium, in the form of Voltaren K Migraine 50 mg coated tablets, is prescribed for migraines.

A potentiation or possible combination effect of diazepam and diclofenac is in any case not common knowledge, nor is it suggested by the known mechanisms of action. Nevertheless, a multitude of references to the two substances in combination can be found in the literature.

Combination effects of muscle relaxants with analgesic nonsteroidal anti-inflammatory drugs, NSAIDs, are already generally described in U.S. Pat. No. 4,923,898 A, a multitude of substances and substance classes being mentioned for each of the two groups. These also include diclofenac and diazepam, but although in principle taken into account, no example with this combination was mentioned, nor was the combination of diclofenac and diazepam explicitly or generically claimed in the claims. It can therefore be assumed that the reinforcing combination effect was not recognized and that the attention of experts in the field should be directed at other medicaments and medicament combinations.

Patent application WO 86/03681 A1 reports on the combination effect with additional caffeine. Here, too, there is no explicit mention of the combination of diclofenac and diazepam, although the two substances were each combined with other muscle relaxants and with analgesic nonsteroidal anti-inflammatory drugs, as well as with caffeine. Thus, not only was the reinforcing combination effect of diclofenac with diazepam not recognized, but also the fact that other additional drugs intended to alleviate the side effects of one of the combined drugs would or could at the same time eliminate the reinforcing effect of the combination.

As a result, a large number of medicaments have been described, which, among many other substances, also contained small added amounts of diclofenac and diazepam; here too, the reinforcing combination effect was not recognized or was unknowingly countered by other constituents of the mixture. At the same time, reports came to light in which mixtures that also contained the two substances gave rise to appreciable side effects. The article “Rutschbahn in den legalen Drogensumpf” [Slippery slope into the mire of legal drugs], Der Spiegel 37/1987, pages 228-229, 232, 235, 238, 241, 244-245, 248-249, 252-253, describes the death of an athlete due to the combination of different painkillers including, among many others, also diazepam and diclofenac.

WO 02/11700 A1 describes an external use of a combination of diazepam and diclofenac, each product being present in the preparation in a content of 1%. The product was intended to be rubbed into the affected area. It is clear that use for joint pain, migraines, and back pain cannot be considered here, since such pain is not caused by superficial muscles and an applied solution would not bring any relief, although Voltaren ointment containing diclofenac as its principal constituent is said to act on joint pain. On the other hand, purely external use also does not give rise to the problem of side effects.

Investigations have also been carried out on the receptors for diclofenac sodium and diazepam, the aim of which however was to measure the displacement by sulfamethoxazole (MDd Kamal Hossain, Amina Khatun, Mahmudur Rahman, Md Nahid Akter, Sadia Afreen Chowdhury, SM Mahbubul Alam: Characterization of the Effect of Drug-Drug-Interaction on Protein Binding in Concurrent Administration of Sulfamethoxazole and Diclofenac Sodium Using Bovine Serum Albumin, Advanced Pharmaceutical Bulletin 2016, 6(4), 589-595). It was found that such a displacement did take place and that at the same time diclofenac sodium also displaced diazepam from the corresponding receptors. Insofar as an interaction was indeed established, no reinforcing of the effect by a combination was found or was suggested by the results.

The combination of diclofenac sodium and diazepam was found to have a satisfactory effect in an Iranian study in surgery to remove uterine polyps (Zahra Asgari, Maryam Razavi, Reihaneh Hosseini, Masoumeh Nataj, Mahroo Rezaeinejad, Mandi Sepidarkish, Evaluation of Paracervical Block and IV Sedation for Pain Management during Hysteroscopic Polypectomy: A Randomized Clinical Trial, Hindawi Pain Research and Management, volume 2017, article ID 5309408, 7 pages). In this study, single doses of 10 mg diazepam and 100 mg diclofenac were administered and this was compared with a comparison group having conventional anesthesia. No significant differences between the comparison groups were found and no side effects occurred. A comparable effect was thus observed.

A combination of diazepam and diclofenac is used rectally in preparation for surgery in young children. A comparative study (S. M. Filatov, G. A. Baer, M. G. F. Rorarius, M. Oikkonen: Efficacy and safety of premedication with oral ketamine for day-case adenoidectomy compared with rectal diazepam/diclofenac and EMLA, Acta Anaesthesiol Scand 2000, 44, 118-124) showed no significant difference versus administration of ketamine. Since there was hardly any preoperative pain to be controlled, synergy effects were not observable.

In a study investigating the effects of a combination of rectally administered diclofenac and nabumetone, both analgesic nonsteroidal anti-inflammatory drugs (NSAIDs), diazepam was used as a comparison (Nao Setoguchi, Norito Takamura, Ken-ichi Fujita, Kenji Ogata, Jin Tokunaga, Toyotaka Nishio, Etsuo Chosa, Kazuhiko Arimori, Keiichi Kawai, Ryuichi Yamamoto: A diclofenac suppository-nabumetone combination therapy for arthritic pain relief and a monitoring method for the diclofenac binding capacity of HSA site II in rheumatoid arthritis; Biopharmaceutics & Drug Disposition 34, 125-136 (2013)). Although diclofenac was found to bind to site II of the human serum protein HSA and not to site I, it was found that diazepam and diclofenac compete for this binding and that diazepam displaces diclofenac. The consequence of this is that the blood concentration of free diclofenac rises again, since it is not firmly bound by the HSA, which makes it better able to penetrate tissues in order to exert its effect. However, the study does not identify an interaction between diclofenac and diazepam, but does conclude that the method is effective in observing HSA binding when a combination of diclofenac with nabumetone is used.

This exertion of the effect of diclofenac with an increase in its blood concentration is not however a given, since diazepam could equally be displacing the active substance diclofenac from other receptors with a similar structure to HSA site II. Although diclofenac would then be able to accumulate better in the tissue, this would not result in a better mechanism of action. The inhibition of cyclooxygenase and thus of reduced prostaglandin synthesis would thus be neither potentiated nor more selective.

The release of diclofenac due to diazepam also affects metabolism. Free diclofenac, like free diazepam, is broken down more rapidly provided the breakdown mechanism is not also blocked. The rate-determining breakdown step for diclofenac is determined by the enzyme CYP2C9, for diazepam it is CYP2C19, both of which are enzymes of the cytochrome P450 enzyme system. Mutual influencing does not appear to occur here. It should however be emphasized that a large number of other common medicaments and some foods can have a considerable influence here. This applies for example to commercially available proton-pump blockers, which are used to counter the digestive complications of diclofenac, and also to antidepressants, drugs that modify blood pressure, and other NSAIDs. It is therefore advisable to take no other medicaments or to temporarily suspend their use under medical supervision.

Pain is transmitted by nerves to the brain by pain sensors known as nociceptors, which are located almost everywhere in the body, for example in the skin, entrails or blood vessels. The pain receptors react when it gets too hot or too cold, or when a certain pressure is exceeded, or when there is chemical irritation, for example when chemical substances are released during inflammation.

The object of the invention is therefore to provide a new pain medicament for symptomatic control and lasting cure of moderate to very severe pain.

Pain is in the practice usually classified on a pain scale from 0 to 10. A pain scale with 10 levels is used hereinbelow for describing pain intensity in accordance with www.ratgeber-schmerzen.de/schmerzen/definition/schmerzskala/; although scales commonly used in different countries are similar, all suffer from a general lack of objective measurability and usually have to be determined subjectively:
0: Pain free.
1: Pain very mild, barely noticeable.
2: Mild pain: a little troubling, perhaps becoming a little stronger from time to time.
3: Noticeable pain: pain that draws attention, but is easy to live with and get used to.
4: Moderate pain: if the patient is distracted, he/she can ignore it for a while, but it is always perceptible.
5: Moderate pain: those affected can ignore it for a few minutes at most and have to make an active effort if they want to concentrate on work or take part in social activities.
6: Moderate pain: disturbs the patient in all normal everyday activities; it is difficult to concentrate.
7: Severe pain: dominates perceptions; social contact and the execution of normal everyday activities are significantly impaired; sleep is also disturbed.
8: Intense pain: physical activity is severely restricted; conversations are possible only with great effort, roughly equivalent to the pain in natural childbirth.
9: Unbearable pain: conversation impossible, the patient screams or moans.
10: Indescribable pain: the patient is unable to stand up and may be delirious.

The invention relates primarily to pain levels 5 to 10, but is also suitable and useful for lower levels of pain if given in a low dose.

It achieves the object of symptomatic control and lasting healing through a combined preparation of the two active ingredients diazepam and diclofenac sodium for the treatment of pain, especially burning and stabbing pain, for the following indications:
herniated disc,
lumbago/back pain,
lumbago with sciatica,
dorsalgia,
neck pain/tension neck pain, cervical spine syndrome,
cervical neuralgia,
sciatica,
facet syndrome,
chronic limb pain,
limb pain in osteoporosis, arthrosis, arthritis, soft tissue rheumatism, tendon injuries, ischemia, obesity,
neuralgia, fibromyalgia, fibromyalgia syndrome,
migraine,
headache,
osteoarthritis and joint pain,
hip pain, knee pain, heel pain, toe pain,
kidney pain,
myocardial infarction,
postoperative pain,
broken bones,
heel spur,
body malpositions,
burns,
tumor pain or tumor-related pain including cancer and metastases,
embolism/thrombosis pain,
rheumatism,
toothache and jaw pain,
pain and depression symptoms during drug withdrawal,
non-localizable pain or pain of unknown origin,
pain caused by fascia and connective tissue,
phantom pain after amputations,
piriformis syndrome,
abdominal pain, both in men and women,
endometriosis,
hemorrhoids,
pain with depression, sleep disorders, anxiety,
burns, scalds, and chemical burns,
combinations of the abovementioned pains.

Since physical pain can also result in psychological pain, this is also reduced accordingly. The suppression of pain is also beneficial in respect of pain memory.

Administration takes place preferably orally in the form of tablets or capsules. However, it can also take the form of an injection, infusion, drops or a drinking ampoule. Each of these forms of administration has advantages and disadvantages; administration as an injection or infusion results in a rapid onset of action, but the effect wears off sooner. Tablets or capsules packaged as prolonged-release tablets can span different periods of action; this principle is already well known with Voltaren Resinat and can be used to advantage with the combination product too. Administration as drops has the advantage of accurate dispensing of single and multiple doses, but care must be taken to ensure that no demixing or flocculation takes place in the embedding liquid, which the pharmacist or the user must take due account of.

In the case of a liquid template, care must be taken to ensure it contains no active substances, just substances that are inert in the body, such as water or propylene glycol, which are able to dissolve diclofenac sodium and diazepam alike.

The dosage depends on the severity of the pain and corresponds to the usual dosages when the individual substances are taken orally or used according to another customary method of administration. The standard dose comprises 5 mg of diazepam and 75 mg of diclofenac sodium. The dose may vary in children, the elderly, and in patients with pre-existing conditions and is from 1 mg to 100 mg diazepam and 1 mg to 150 mg diclofenac sodium.

Short-term use or long-term use is possible. Short-term use is intended to be for a few hours up to a few days. In long-term use, it can be taken for several weeks as prescribed by a physician. For pain of pain severity 1 to 5, a standard dose should be taken once or twice a day, for pain of severity 5 to 8 a standard dose should be taken two to three times per day, and for pain of severity 8 to 10 a standard dose should be taken two to four times per day.

Because of interactions, an interval of at least one hour should be allowed between each dose and dosing with antibiotics.

Study results in individual cases demonstrate the extraordinary effectiveness:
Study 1: Female patient, 47 years old, neck pain,
   Dosage: Single dose of 75 mg diclofenac sodium+5 mg diazepam in tablet form,
   no further doses necessary after relief.
Study 2: Female patient, 47 years old, severe back pain,
   Dosage: Morning and evening doses of 75 mg diclofenac sodium+5 mg diazepam in tablet form for 2 days,
   no further doses necessary after relief.
Study 3: Female patient, 47 years old, endometriosis,
   Dosage: 75 mg diclofenac sodium+5 mg diazepam in tablet form once a day for 2 days,
   no further doses necessary after relief.
Study 4: Male patient, 42 years old, acute neck pain and severe migraines, pain scale 5-10,
   Dosage: 75 mg diclofenac sodium+5 mg diazepam in tablet form three times a day for 3 days,
   no further doses necessary after relief.
Study 5: Male patient, 20 years old, shoulder pain after rotator cuff injury during fitness exercise with shoulder press, pain scale 5-8,
   Dosage: Initially 75 mg diclofenac sodium+5 mg diazepam in tablet form twice daily, reduced to once daily after improvement to 1-5 on the pain scale, total duration one week,
   no further doses necessary thereafter.
Study 6: Male patient, 52 years old, acute intervertebral disc pain, pain scale 8 to 9,
   Dosage: 75 mg diclofenac sodium+5 mg diazepam in tablet form twice daily for 1 week, no further doses necessary thereafter.

In study 6 it was possible to avoid long-term medication with morphine, as had originally been planned.

An advantage of the invention is that it allows the administration of opioids such as morphine to be avoided even in very severe pain of pain severity 8 to 10. Another advantage is that pain can be permanently relieved or eliminated. Long-term use can usually be dispensed with; continuation is then necessary only if symptoms return as a result of stress or a shock.

1. A pharmaceutical composition consisting of a combined preparation of diazepam and diclofenac sodium for use in the specific treatment of pain, in particular burning and stabbing pain, on the basis of the indication, from
herniated disc,
lumbago/back pain,
lumbago with sciatica,
dorsalgia,
neck pain/tension neck pain, cervical spine syndrome,
cervical neuralgia,
sciatica,
facet syndrome,
chronic limb pain,
limb pain in osteoporosis, arthrosis, arthritis, soft tissue rheumatism, tendon injuries, ischemia, obesity,
neuralgia, fibromyalgia, fibromyalgia syndrome,
migraine,
headache,
osteoarthritis and joint pain,
hip pain, knee pain, heel pain, toe pain,
kidney pain,
myocardial infarction,
postoperative pain,
broken bones,
heel spur,
body malpositions,
burns,
tumor pain or tumor-related pain including cancer and metastases,
embolism/thrombosis pain,
rheumatism,
toothache and jaw pain,
pain and depression symptoms during drug withdrawal,
non-localizable pain or pain of unknown origin,
pain caused by fascia and connective tissue,
phantom pain after amputations,
piriformis syndrome,
abdominal pain, both in men and women,
endometriosis,
hemorrhoids,
pain with depression, sleep disorders, anxiety,
burns, scalds, and chemical burns,
combinations of the abovementioned pains.

2. The pharmaceutical composition for use as claimed in claim 1 in the form of tablets, capsules, injection, infusion, drinking ampoules or drops.

3. The pharmaceutical composition for use as claimed in claim 1 wherein by a single dose of from 1 mg to 100 mg diazepam and 1 mg to 150 mg diclofenac sodium.

4. The pharmaceutical composition for use as claimed in claim 3, wherein by a single dose of 5 mg diazepam and 75 mg diclofenac sodium.
